SEQUENCE LISTING (52 sequences)

SEQ ID NO: 1 - DNA, 35 bp, Homo sapiens
aatgaatgaatgaatgaatgatgaatgaatgaatg

SEQ ID NO: 2 - DNA, 83 bp, Unknown
  Description of Unknown Illustrative oligonucleotide
gcagacccataatgtcaatagatgtggtagaagtcgtcatttggcgagaaagctcagtcccaggaggaagcggagcagtccaa

SEQ ID NO: 3 - DNA, 83 bp, Unknown
  Description of Unknown Illustrative oligonucleotide
ttccaatcatagccacagtttacaacatttgtatctttatctgtatccttatttatacatctatctatctatctatctatcta

SEQ ID NO: 4 - DNA, 83 bp, Unknown
  Description of Unknown Illustrative oligonucleotide
cttcctactggcccataatccagctgtgggagggagccagtggatttggaaaccgacatggcttggccttgcctgcctgcctc

SEQ ID NO: 5 - DNA, 157 bp, Unknown
  Description of Unknown Illustrative polynucleotide
ccagggcagtgttccaacctgagtctgccaaggcctagcaggttgctaaccaccctgtgtctcagttttcctacctgtaaaatgaagatattaccagtaactgccttcatagatagaagatagatagattagatggatagatagatagatagataga

SEQ ID NO: 6 - DNA, 157 bp, Unknown
  Description of Unknown Illustrative polynucleotide
ccagggcagtgttccaacctgagtctgccaaggactagcaggttgctaaccaccctgtgtctcagttttcctacctgtaaaatgaagatattaacagtaactgccttcatagatagaagatagatagattagatagttagatagatagatagataga

SEQ ID NO: 7 - DNA, 157 bp, Unknown
  Description of Unknown Illustrative polynucleotide
ccagggcagtgttccaacctgagtctgccaaggactagcaggttgctaaccaccctgtgtctcagttttcctacctgtaaaatgaagatattaacagtaactgccttcatagatagaagatagatagattagatagatagatagatagatagataga

SEQ ID NO: 8 - DNA, 40 bp, Unknown
  Description of Unknown Illustrative oligonucleotide
agatagatagatagatagatagatagatagatagatagat

SEQ ID NO: 9 - DNA, 52 bp, Unknown
  Description of Unknown Illustrative oligonucleotide
agatagatagatagatagatagatagatagatagatagatagatagatagat

SEQ ID NO: 10 - DNA, 48 bp, Unknown
  Description of Unknown Illustrative oligonucleotide
agatagatagatagatagatagatagatagatagatagatagatagat

SEQ ID NO: 11 - DNA, 36 bp, Unknown
  Description of Unknown Illustrative oligonucleotide
agatagatagatagatagatagatagatagatagat

SEQ ID NO: 12 - DNA, 20 bp, Unknown
  Description of Unknown Illustrative oligonucleotide
agatagatagatagatagat

SEQ ID NO: 13 - DNA, 107 bp, Unknown
  Description of Unknown Illustrative polynucleotide
tctatctatctatctatctgtctgtctgtctgtctgtctgtctatctatctatatctatctatctatcatctatctatccatatctatctatctatctatctatcta

SEQ ID NO: 14 - DNA, 12 bp, Unknown
  Description of Unknown Illustrative oligonucleotide
tctatctatcta

SEQ ID NO: 15 - DNA, 16 bp, Unknown
  Description of Unknown Illustrative oligonucleotide
tctatctatctatcta

SEQ ID NO: 16 - DNA, 20 bp, Unknown
  Description of Unknown Illustrative oligonucleotide
tctatctatctatctatctg

SEQ ID NO: 17 - DNA, 24 bp, Unknown
  Description of Unknown Illustrative oligonucleotide
tctatctatctatctatctgtctg

SEQ ID NO: 18 - DNA, 28 bp, Unknown
  Description of Unknown Illustrative oligonucleotide
tctatctatctatctatctgtctgtctg

SEQ ID NO: 19 - DNA, 32 bp, Unknown
  Description of Unknown Illustrative oligonucleotide
tctatctatctatctatctgtctgtctgtctg

SEQ ID NO: 20 - DNA, 36 bp, Unknown
  Description of Unknown Illustrative oligonucleotide
tctatctatctatctatctgtctgtctgtctgtctg

SEQ ID NO: 21 - DNA, 40 bp, Unknown
  Description of Unknown Illustrative oligonucleotide
tctatctatctatctatctgtctgtctgtctgtctgtctg

SEQ ID NO: 22 - DNA, 44 bp, Unknown
  Description of Unknown Illustrative oligonucleotide
tctatctatctatctatctgtctgtctgtctgtctgtctgtcta

SEQ ID NO: 23 - DNA, 48 bp, Unknown
  Description of Unknown Illustrative oligonucleotide
tctatctatctatctatctgtctgtctgtctgtctgtctgtctatcta

SEQ ID NO: 24 - DNA, 52 bp, Unknown
  Description of Unknown Illustrative oligonucleotide
tctatctatctatctatctgtctgtctgtctgtctgtctgtctatctatcta

SEQ ID NO: 25 - DNA, 53 bp, Unknown
  Description of Unknown Illustrative oligonucleotide
tctatctatctatctatctgtctgtctgtctgtctgtctgtctatctatctat

SEQ ID NO: 26 - DNA, 54 bp, Unknown
  Description of Unknown Illustrative oligonucleotide
tctatctatctatctatctgtctgtctgtctgtctgtctgtctatctatctata

SEQ ID NO: 27 - DNA, 58 bp, Unknown
  Description of Unknown Illustrative oligonucleotide
tctatctatctatctatctgtctgtctgtctgtctgtctgtctatctatctatatcta

SEQ ID NO: 28 - DNA, 62 bp, Unknown
  Description of Unknown Illustrative oligonucleotide
tctatctatctatctatctgtctgtctgtctgtctgtctgtctatctatctatatctatcta

SEQ ID NO: 29 - DNA, 66 bp, Unknown
  Description of Unknown Illustrative oligonucleotide
tctatctatctatctatctgtctgtctgtctgtctgtctgtctatctatctatatctatctatcta

SEQ ID NO: 30 - DNA, 67 bp, Unknown
  Description of Unknown Illustrative oligonucleotide
tctatctatctatctatctgtctgtctgtctgtctgtctgtctatctatctatatctatctatctat

SEQ ID NO: 31 - DNA, 68 bp, Unknown
  Description of Unknown Illustrative oligonucleotide
tctatctatctatctatctgtctgtctgtctgtctgtctgtctatctatctatatctatctatctatc

SEQ ID NO: 32 - DNA, 69 bp, Unknown
  Description of Unknown Illustrative oligonucleotide
tctatctatctatctatctgtctgtctgtctgtctgtctgtctatctatctatatctatctatctatca

SEQ ID NO: 33 - DNA, 73 bp, Unknown
  Description of Unknown Illustrative oligonucleotide
tctatctatctatctatctgtctgtctgtctgtctgtctgtctatctatctatatctatctatctatcatcta

SEQ ID NO: 34 - DNA, 77 bp, Unknown
  Description of Unknown Illustrative oligonucleotide
tctatctatctatctatctgtctgtctgtctgtctgtctgtctatctatctatatctatctatctatcatctatcta

SEQ ID NO: 35 - DNA, 81 bp, Unknown
  Description of Unknown Illustrative oligonucleotide
tctatctatctatctatctgtctgtctgtctgtctgtctgtctatctatctatatctatctatctatcatctatctatcca

SEQ ID NO: 36 - DNA, 82 bp, Unknown
  Description of Unknown Illustrative oligonucleotide
tctatctatctatctatctgtctgtctgtctgtctgtctgtctatctatctatatctatctatctatcatctatctatccat

SEQ ID NO: 37 - DNA, 83 bp, Unknown
  Description of Unknown Illustrative oligonucleotide
tctatctatctatctatctgtctgtctgtctgtctgtctgtctatctatctatatctatctatctatcatctatctatccata

SEQ ID NO: 38 - DNA, 87 bp, Unknown
  Description of Unknown Illustrative oligonucleotide
tctatctatctatctatctgtctgtctgtctgtctgtctgtctatctatctatatctatctatctatcatctatctatccatatcta

SEQ ID NO: 39 - DNA, 91 bp, Unknown
  Description of Unknown Illustrative oligonucleotide
tctatctatctatctatctgtctgtctgtctgtctgtctgtctatctatctatatctatctatctatcatctatctatccatatctatcta

SEQ ID NO: 40 - DNA, 95 bp, Unknown
  Description of Unknown Illustrative oligonucleotide
tctatctatctatctatctgtctgtctgtctgtctgtctgtctatctatctatatctatctatctatcatctatctatccatatctatctatcta

SEQ ID NO: 41 - DNA, 99 bp, Unknown
  Description of Unknown Illustrative oligonucleotide
tctatctatctatctatctgtctgtctgtctgtctgtctgtctatctatctatatctatctatctatcatctatctatccatatctatctatctatcta

SEQ ID NO: 42 - DNA, 103 bp, Unknown
  Description of Unknown Illustrative polynucleotide
tctatctatctatctatctgtctgtctgtctgtctgtctgtctatctatctatatctatctatctatcatctatctatccatatctatctatctatctatcta

SEQ ID NO: 43 - DNA, 60 bp, Unknown
  Description of Unknown Illustrative oligonucleotide
tttctttctttcttttttctctttctttctttctttctttctttctttctccttccttcc

SEQ ID NO: 44 - DNA, 64 bp, Unknown
  Description of Unknown Illustrative oligonucleotide
tttctttctttcttttttctctttctttctttctttctttctttctttctttctccttccttcc

SEQ ID NO: 45 - DNA, 65 bp, Unknown
  Description of Unknown Illustrative oligonucleotide
tttctttctttcttttttctctttctttctttctttcttttctttctttctttctccttccttcc

SEQ ID NO: 46 - DNA, 66 bp, Unknown
  Description of Unknown Illustrative oligonucleotide
tttctttctttcttttttctttctttctttctttctttctttctttctttctttctccttccttcc

SEQ ID NO: 47 - DNA, 68 bp, Unknown
  Description of Unknown Illustrative oligonucleotide
tttctttctttcttttttctctttctttctttctttctttctttctttctttctttctccttccttcc

SEQ ID NO: 48 - DNA, 72 bp, Unknown
  Description of Unknown Illustrative oligonucleotide
tttctttctttcttttttctctttctttctttctttctttctttctttctttctttctttctccttccttcc

SEQ ID NO: 49 - DNA, 74 bp, Unknown
  Description of Unknown Illustrative oligonucleotide
tttctttctttcttttttctttctttctttctttctttctttctttctttctttctttctttctccttccttcc

SEQ ID NO: 50 - DNA, 76 bp, Unknown
  Description of Unknown Illustrative oligonucleotide
tttctttctttcttttttctctttctttctttctttctttctttctttctttctttctttctttctccttccttcc

SEQ ID NO: 51 - DNA, 78 bp, Unknown
  Description of Unknown Illustrative oligonucleotide
tttctttctttcttttttctttctttctttctttctttctttctttctttctttctttctttctttctccttccttcc

SEQ ID NO: 52 - DNA, 80 bp, Unknown
  Description of Unknown Illustrative oligonucleotide
tttctttctttcttttttctctttctttctttctttctttctttctttctttctttctttctttctttctccttccttcc

(19) United States
(12) Patent Application Publication
(10) Pub. No.: US 2020/0135297 A1
(43) Pub. Date: Apr. 30, 2020

(54) Computer Files and Methods Supporting Forensic Analysis of Nucleotide Sequence Data

(71) Applicant: Battelle Memorial Institute, Columbus, OH (US)

(72) Inventors: Brian A. Young, Columbus, OH (US); Angela T. Minard-Smith, Marysville, OH (US); Esley M. Heizer, JR., Galloway, OH (US)

(21) Appl. No.: 16/654,094

(22) Filed: Oct. 16, 2019

Related U.S. Application Data
(62) Division of application No. 13/834,830, filed on Mar. 15, 2013, now abandoned.

(51) Int. Cl. G16B 30/00

(52) U.S. Cl. CPC G16B 30/00

(57) ABSTRACT

In one illustrative embodiment, an allelotyping method may comprise using a massively parallel sequencing (MPS) instrument to read nucleotide sequences in a sample and to generate nucleotide sequence data quantifying each read of a nucleotide sequence in the sample, determining, for each read by the MPS instrument, whether a portion of the generated nucleotide sequence data represents a short tandem repeat (STR) associated with a corresponding locus, adding each portion of the nucleotide sequence data determined to represent an STR to a locus-specific list for the corresponding locus, determining, for each locus-specific list, a number of occurrences within that locus-specific list of identical nucleotide sequence data representing a unique STR, and identifying each unique STR for which the number of occurrences of identical nucleotide sequence data within the locus-specific list exceeds an abundance threshold as an allele of the corresponding locus for the sample.

### CROSS-REFERENCE TO RELATED APPLICATION

This application is a divisional of U.S. patent application Ser. No. 13/834,830, filed Mar. 15, 2013, the entire disclosure of which is incorporated herein by reference.

### SEQUENCE LISTING

This application contains a Sequence Listing which has been submitted in ASCII format via EFS-Web and is hereby incorporated by reference in its entirety. Said ASCII copy, created on Jan. 13, 2020, is named 920006-301651_SL.txt and is 14,038 bytes in size.

### TECHNICAL FIELD

The present disclosure relates, generally, to nucleotide sequence data and, more particularly, to computer files and methods supporting forensic analysis of nucleotide sequence data.

### BACKGROUND

Polymorphic tandem repeats of nucleotide sequences are found throughout the human genome, and the particular combinations of allelic states at multiple repeat sites are sufficiently unique to an individual that these repeating sequences can be used in human or other organism identification. These markers are also useful in genetic mapping and linkage analysis, where the tandem repeat sites may be linked to sites important for determining, for example, predisposition for disease. Tandem repeats can be used directly in human identity testing, such as in forensics analysis. There are many types of tandem repeats of nucleic acids, all falling under the general term variable number tandem repeats (VNTR). These minisatellites and microsatellites are also called short tandem repeats (STR).

One application of tandem repeat analysis is in forensics or human identity testing. In current forensics analyses, highly polymorphic STRs are identified using a DNA sample from an individual and DNA amplification steps, such as polymerase chain reaction (PCR), to provide amplified samples of partial DNA sequences, or amplicons, from the individual's DNA. The amplicons can then be matched by size (i.e., repeat numbers) to reference databases, such as the sequences stored in national or local DNA databases. For example, amplicons that originate from STR loci can be matched to reference STR databases, including the FBI CODIS database in the United States, or the NDNAD database in Europe, to identify the individual by matching to the STR alleles specific to that individual.

Forensic DNA analysis is about to cross a threshold where DNA samples will begin to be analyzed routinely by massively parallel sequencing (MPS), also sometimes referred to in the art as next-generation sequencing. The advent of routine MPS for forensic DNA analysis will create large quantities of nucleotide sequence data that may enable richer exploitation of DNA in forensic applications. Once information is generated on the genetic profile of an individual (e.g., for either forensic investigative purposes or confirmatory matching), the resulting nucleotide sequence data should be formatted for exchange among law enforcement entities. However, no uniform, workable standards for nucleotide sequence data storage and exchange suitable for law enforcement applications currently exist.

In addition to the foregoing storage and exchange problem, forensic analysis requires the preservation of data, including raw data, for evidentiary purposes. In conventional capillary-electrophoresis (CE) workflows, the raw data typically preserved as evidence includes raw nucleotide sequence data (e.g., “.hid” or “.fsa” files) and an image of the electropherogram (e.g., printed or a graphics file). This evidence is routinely provided during the jurisprudence discovery process. The forensic results can be reproduced from “.hid” or “.fsa” files using appropriate software applications. Alternatively, these results can be read directly from the electropherograms by persons practiced in the art. The “.hid” and “.fsa” files produced by typical CE workflows are relatively small and can be easily transmitted or stored. The electropherogram graphics are also small enough to be easily transferred or stored. By contrast, data files created by MPS workflows are typically larger than 1 GB making them difficult to transmit or store. In addition, these files, while text-based, are not human-readable in any practical sense because of their large size. Thus, other human readable forms of the raw data from MPS workflows are needed.

### SUMMARY

According to one aspect, a method may comprise receiving a first text-based computer file including one or more records, each of the one or more records comprising nucleotide sequence data generated by a read of a massively parallel sequencing (MPS) instrument, determining, for each of the one or more records of the first text-based file, whether a portion of the nucleotide sequence data of the record represents a short tandem repeat (STR) associated with a locus, placing each portion of the nucleotide sequence data determined to represent an STR associated with a locus into one of a number of locus-specific lists, determining, for each of the locus-specific lists, a number of occurrences within the locus-specific list of identical nucleotide sequence data representing a unique STR, and generating a second text-based computer file including one or more records, each of the one or more records corresponding to a unique STR for which the number of occurrences of identical nucleotide sequence data representing the unique STR exceeded an abundance threshold.

In some embodiments, the first text-based computer file may be formatted as a FASTQ file. Determining whether a portion of the nucleotide sequence data of a record represents an STR associated with a locus may comprise determining whether a portion of the nucleotide sequence data of the record represents a primer sequence used to amplify the locus. Determining whether a portion of the nucleotide sequence data of the record represents a primer sequence used to amplify the locus may comprise referencing an updateable library of primer sequences. Placing each portion of the nucleotide sequence data determined to represent an STR associated with a locus into one of a number of locus-specific lists may comprise removing a portion of the nucleotide sequence data representing a flanking sequence. Removing a portion of the nucleotide sequence data representing a flanking sequence may comprise referencing an updateable library of flanking sequences. The abundance threshold may be user-defined.

Generating the second text-based computer file may comprise, for each of the one or more records, writing nucleotide sequence data representing the corresponding unique STR to a second text line of the record. Generating the second text-based computer file may further comprise, for each of the one or more records, writing average quality scores for the nucleotide sequence data representing the corresponding unique STR to a fourth text line of the record. The average quality scores may be formatted as average Phred quality scores. Generating the second text-based computer file may further comprise, for each of the one or more records, writing forensic metadata associated with the nucleotide sequence data representing the corresponding unique STR to a first text line of the record. The forensic metadata may be copied from the first text-based computer file. Generating the second text-based computer file may further comprise, for each of the one or more records, writing an attribute-value pair specifying the number of occurrences of identical nucleotide sequence data representing the corresponding unique STR to a third text line of the record.

In some embodiments, generating the second text-based computer file may further comprise, for each of the one or more records, writing a human-readable sequence-based allele (HRSBA) designation that is deterministic of the corresponding unique STR to a third text line of the record. Generating the HRSBA designation may comprise reading a plurality of nucleotide bases in a sliding window that moves along the nucleotide sequence data, determining whether the plurality of nucleotide bases corresponds to a canonical motif of a locus associated with the corresponding unique STR, adding the plurality of nucleotide bases to the HRSBA in response to determining that the plurality of nucleotide bases corresponds to a canonical motif of the locus and represents a first instance of the canonical motif, and moving the sliding window by a plurality of positions in response to determining that the plurality of nucleotide bases corresponds to a canonical motif of the locus.

In some embodiments, generating the HRSBA designation may further comprise adding only a first nucleotide base of the plurality of nucleotide bases to the HRSBA in response to determining that the plurality of nucleotide bases does not correspond to a canonical motif of the locus and moving the sliding window by one position in response to determining that the plurality of nucleotide bases does not correspond to a canonical motif of the locus. The sliding window may move along the nucleotide sequence data in a 5′ to 3′ direction. Determining whether the plurality of nucleotide bases corresponds to a canonical motif of a locus associated with the corresponding unique STR may comprise referencing an updatable library of canonical motifs of one or more loci. Generating the HRSBA designation may further comprise determining whether a final plurality of nucleotide bases of the nucleotide sequence corresponds to a canonical ending motif of a locus associated with the corresponding unique STR and generating a user alert in response to determining that the final plurality of nucleotide bases does not correspond to a canonical ending motif of the locus. Generating the HRSBA designation may comprise referencing an updatable library associating common nucleotide sequences with corresponding HRSBA designations.

According to another aspect, a method may comprise receiving forensic metadata associated with nucleotide sequence data generated by a massively parallel sequencing (MPS) instrument and writing the forensic metadata to a text-based computer file comprising the nucleotide sequence data. In some embodiments, receiving the forensic metadata may comprise receiving data from a case management system of a laboratory operating the MPS instrument. The text-based computer file comprising the nucleotide sequence data may include one or more records, each of the one or more records representing a read of the MPS instrument. The text-based computer file comprising the nucleotide sequence data may be formatted as a FASTQ file.

In some embodiments, writing the forensic metadata to the text-based computer file may comprise writing the forensic metadata to a first text line of each of the one or more records of the text-based computer file. The first text line of each of the one or more records of the text-based computer file may comprise a unique sequence identifier created by the MPS instrument when generating the nucleotide sequence data. Writing the forensic metadata to the first text line of each of the one or more records of the text-based computer file may comprise appending the forensic metadata to the unique sequence identifier. Writing the forensic metadata to the text-based computer file may comprise writing one or more attribute-value pairs to the text-based computer file, each of the one or more attribute-value pairs specifying one of a file format, a unique case identifier, a unique sample identifier, a unique laboratory identifier, and a unique technician identifier.

According to yet another aspect, a computer-readable medium may storing a text-based file comprising one or more records, each of the one or more records including a first text line comprising forensic metadata, a second text line comprising a number of characters representing nucleotide sequence data, a third text line, and a fourth text line comprising a number of characters representing quality scores associated with the nucleotide sequence data.

In some embodiments, each of the characters of the second text line may represent an output of a base call algorithm performed by a massively parallel sequencing (MPS) instrument. The first text line may further comprise a unique sequence identifier created by a massively parallel sequencing (MPS) instrument when generating the nucleotide sequence data. The forensic metadata of the first text line may comprise one or more attribute-value pairs, each of the one or more attribute-value pairs specifying one of a file format, a unique case identifier, a unique sample identifier, a unique laboratory identifier, and a unique technician identifier.

In some embodiments, the nucleotide sequence data may represent a short tandem repeat (STR) for which a read count from a sample exceeded an abundance threshold. The third text line may comprise an attribute-value pair specifying the read count of the STR. Each of the characters of the fourth text line may represent an average quality score associated with a corresponding character of the second text line, the average quality score being a function of quality scores associated with all reads of the STR. Each of the characters of the fourth text line may be formatted as an average Phred quality score. The third text line may comprise one or more attribute-value pairs, each of the one or more attribute-value pairs specifying one of a locus of the STR, a strand of the STR, an analytic threshold associated with the STR, and a designation of the STR as corresponding to one of an allele, a stutter, and an artifact.

In some embodiments, the third text line may comprise a human-readable sequence-based allele (HRSBA) designation that is deterministic of the STR. The HRSBA designation may comprise a first attribute-value pair summarizing the STR in a 5′ to 3′ direction. The first attribute-value pair of the HRSBA designation may comprise one or more integers each followed by one or more characters, each of the one or more integers representing a nucleotide position and each of the one or more characters representing a nucleotide base. The one or more characters following each of the one or more integers in the first attribute-value pair of the HRSBA designation may be bracketed if the one or more characters correspond to a repeat motif of the STR. Each of the characters in the first attribute-value pair of the HRSBA designation may be an International Union of Pure and Applied Chemistry (IUPAC) nucleotide base code. The HRSBA designation may comprise a second attribute-value pair specifying a length of the STR.

### BRIEF DESCRIPTION OF THE DRAWINGS

The concepts described in the present disclosure are illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements. The detailed description particularly refers to the accompanying figures in which:

FIG. 1 is a simplified flow diagram illustrating one embodiment of a method of creating a computer file comprising raw nucleotide sequence data and forensic metadata;

FIG. 2 illustrates a portion of one embodiment of a text-based computer file comprising raw nucleotide sequence data (SEQ ID NOS 2-4, respectively, in order of appearance);

FIG. 3 illustrates a portion of one embodiment of a text-based computer file comprising raw nucleotide sequence data (SEQ ID NOS 5-7, respectively, in order of appearance) and forensic metadata;

FIG. 4 is a simplified flow diagram illustrating one embodiment of a method of creating a computer file comprising summary nucleotide sequence data and metadata;

FIG. 5 illustrates a portion of one embodiment of a text-based computer file comprising summary nucleotide sequence data (SEQ ID NOS 8-11, respectively, in order of appearance) and metadata;

FIG. 6 illustrates pseudo-code for a motif aware allellotyper (MAA) algorithm;

FIG. 7 graphically illustrates one example of the operation of the MAA algorithm of FIG. 6 (FIG. 7 discloses the top sequence as SEQ ID NO: 12, “1[AGAT]5 . . . 17[AGAT]21” as SEQ ID NO: 12 and “1[AGAT]5 . . . 1[AGAT]” as SEQ ID NO: 12);

FIG. 8 graphically illustrates another example of the operation of the MAA algorithm of FIG. 6 (FIG. 8 discloses the sequences in the middle column as SEQ ID NOS 13 and 13 and the sequences in the right column, beginning with “1[TCTA]13,” as SEQ ID NOS 14-42 and 13, respectively, in order of appearance);

FIG. 9 illustrates a portion of one embodiment of a look-up table that may be contained in an updatable library (FIG. 9(d) discloses SEQ ID NOS 43, 43-44, 44-45, 45-46, 46-47, 47-48, 48-49, 49-50, 50-51, 51-52 and 52, respectively, in order of appearance); and

FIG. 10 is a simplified flow diagram illustrating one embodiment of an allelotyping method.

### DETAILED DESCRIPTION OF THE DRAWINGS

While the concepts of the present disclosure are susceptible to various modifications and alternative forms, specific embodiments thereof have been shown by way of example in the figures and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

The disclosed embodiments may be implemented, in some cases, in hardware, firmware, software, or any combination thereof. The disclosed embodiments may also be implemented as instructions carried by or stored on a transitory or non-transitory computer-readable storage medium, which may be read and executed by one or more processors. A computer-readable storage medium may be embodied as any storage device, mechanism, or other physical structure for storing or transmitting information in a form readable by a computing device (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

The present disclosure relates to a new computer file format useful for storing, exchanging, and/or preserving evidence regarding nucleotide sequence data, including short tandem repeat (STR) profiling data, particularly when the data originates from a massively parallel sequencing (MPS) instrument or workflow. As used herein, the term “nucleotide sequence data” generally refers to symbolic representations of nucleotides arranged in sequential fashion. This new computer file format, which is illustratively referred to herein as the “FASTF” file format, accommodates the conventions and requirements of MPS-derived data, as well as the conventions and requirements of evidence preservation in forensic DNA analysis. For example, MPS-derived data typically conforms to the following conventions, among others: (1) use of text-based computer files for data exchange, (2) storage of the raw nucleotide sequence data, (3) storage of per-nucleotide quality scores in Phred format, (4) storage of sequencer-derived metadata, and (5) traceability of data to the level of individual reads. Forensic DNA analysis may require (1) recording of information necessary for evidence traceability, including case management metadata, (2) expression of STR alleles in a human-readable format, and (3) translation of allele lengths into a CODIS-compatible format. In at least some embodiments, the FASTF file format addresses each of the foregoing concerns.

The FASTF file format achieves compatibility with MPS workflows by incorporating certain aspects of the FASTQ file format that is output by many MPS instruments (and, thus, currently serves as a de facto industry standard). First, the FASTF file format uses a text-based format that allows human readability. Second, the FASTF file format uses a repeating four-row record convention. Unlike FASTQ, however, the FASTF file format supports the evidentiary requirements inherent to forensic DNA analysis through the addition of certain metadata content.

In some illustrative embodiments, a FASTF file may conform to the file format specification outlined in Table 1 and further described below. Table 1 sets forth a FASTF file format specification with reference to the FASTQ file (the third column denoting several distinguishing features of FASTF files, as compared to FASTQ files). It will be appreciated that, in other embodiments, a FASTF file may be formatted according to additional or different specifications than those set forth in Table 1.

TABLE 1

| Line Type | FASTQ | FASTF (Differences from FASTQ) |
|---|---|---|
| 1) Title and | First character must | The systematic identifier produced |
| Description | be “@” | by MPS instrument/software may |
| | Free format field with | comprise the initial portion of |
| | no length limitation | this field |
| | Arbitrary content can | Forensic metadata as a series of |
| | be included | optional attribute-value pairs |
| 2) Sequence | No specific initial | Only International Union of Pure |
| | character is required | and Applied Chemistry (IUPAC) |
| | for this line type | nucleotide base codes are |
| | Any printable | permitted |
| | character is permitted | (“ACTGNURYSWKMBDHVN.-”) |
| | Upper case, lower | |
| | case, and mixed case | |
| | accepted | |
| | Can be line wrapped | |
| 3) End of | First character must | Content after “+” does not |
| Sequence | be “+” | have to match the title line |
| | If the title line is | STR metadata as a series of |
| | repeated, it must be | optional attribute-value pairs |
| | identical | |
| 4) Quality | Accepts printable | |
| Scores | ASCII characters | |
| | 33-126 | |
| | Can be line wrapped | |
| | Length must be equal | |
| | to sequence length | |

Similar to FASTQ files, a FASTF file may comprise one or more records, where each record includes data in four text lines. The first and third text lines of a record may contain metadata related to case management and to evidence preservation. The second text line contains nucleotide sequence data, while the fourth text line contains quality scores for the nucleotide calls represented by the second text line. In the illustrative embodiment, the quality scores of the fourth text line are formatted as Phred quality scores. FASTF files may contain either raw nucleotide sequence data or summary nucleotide sequence data and meta data (e.g., created by an allelotyping process). As described further below, an attribute-value pair within the first text line of each record may indicate whether a particular file contains raw or summary data and metadata.

The present disclosure also relates to methods of creating and/or processing FASTF files. For instance, a pair of algorithms (illustratively referred to in the present disclosure as “FASTF Creator Raw” or “F_CR” and as “FASTF Creator Summary” or “F_CS,” respectively) may be used to create FASTF files containing raw or summary data and metadata. The FASTF Creator Raw and FASTF Creator Summary algorithms are designed for use at different stages of the forensic DNA analysis process. In some embodiments, these two FASTF file creation algorithms may be complemented by a number of utility algorithms that convert FASTF files to other formats and otherwise manipulate FASTF files (illustratively referred to herein as “FASTFtools”).

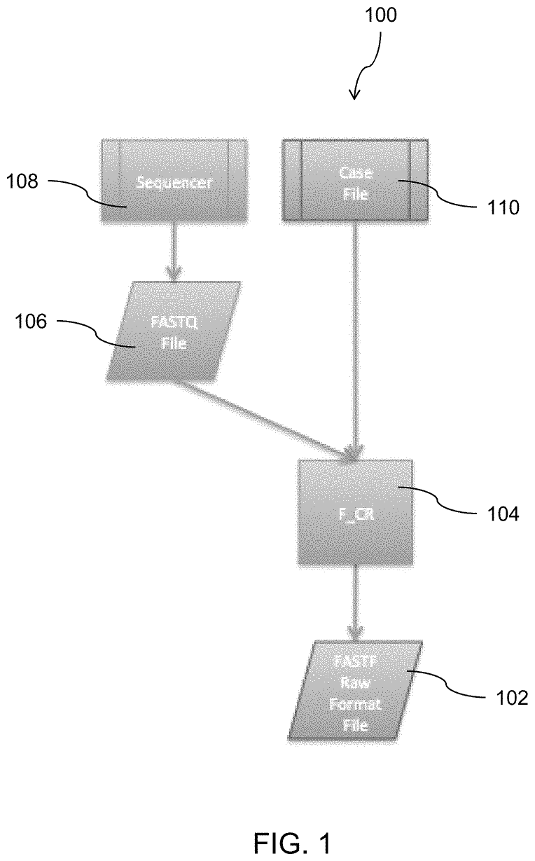

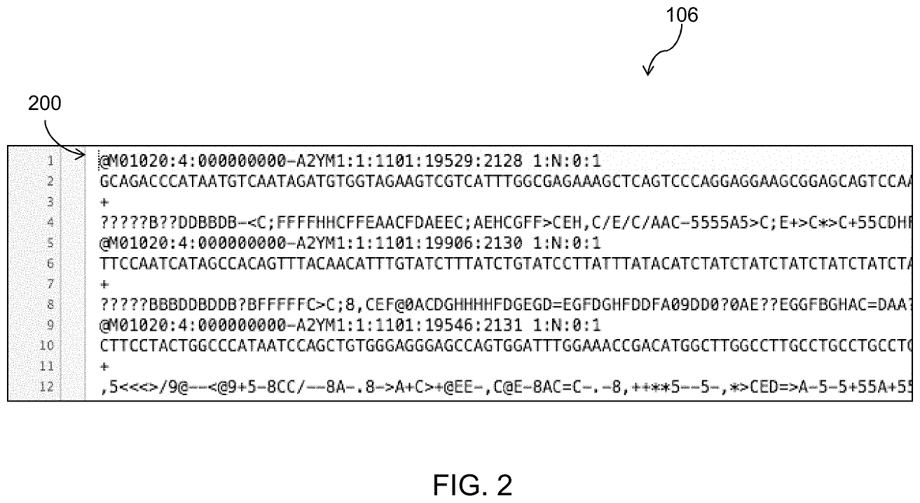

Referring now to FIG. 1, a method 100 of creating FASTF files containing raw nucleotide sequence data and forensic metadata is shown is as a simplified flow diagram. The method 100 is intended for use near the beginning of the forensic DNA analysis process to create a FASTF file 102 that may serve as an evidentiary record comprising both raw nucleotide sequence data and case management information. As shown in FIG. 1, the F_CR algorithm 104 may ingest data from two different sources, among others. Raw nucleotide sequence data, quality scores associated with the nucleotide sequence data, and instrument-specific metadata may be ingested from a FASTQ file 106 generated by an MPS instrument 108 (and its associated software). The first twelve lines (i.e., three records) of one illustrative example of a FASTQ file 106, output from an Illumina MiSeq MPS instrument, are shown in FIG. 2. As shown in FIG. 2, a first text line of each record of the FASTQ file 106 includes a unique sequence identifier 200 created by the MPS instrument 108, a second text line of each record of the FASTQ file 106 comprises a number of characters representing nucleotide sequence data, and a fourth text line of each record of the FASTQ file 106 comprises a number of characters representing quality scores associated with the nucleotide sequence data. Each record of the FASTQ file 106 represents a read of the MPS instrument 108, and each of the characters of the second text line represents an output of a base call algorithm performed by the MPS instrument 108.

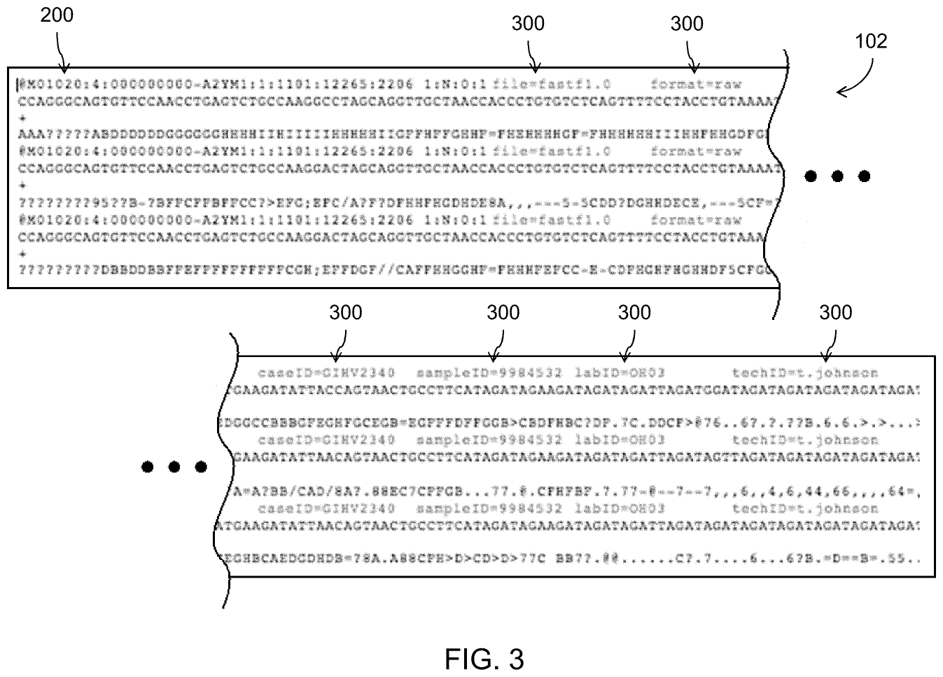

The F_CR algorithm 104 may also ingest a case file 110 received from a laboratory case management system. In other embodiments of the method 100, the case management information may be manually input by a user of the MPS instrument via a user interface. Using these inputs, the F_CR algorithm 104 may write forensic metadata to each record of contained in the FASTQ file 106 a text-based computer file comprising the nucleotide sequence data. The output of the F_CR algorithm 104 is a FASTF raw-format file 102, one illustrative example of which is shown in FIG. 3 (showing the first twelve lines, i.e. three records, of the file 102). In some embodiments, the FASTF raw-format file 102 may include all of the data contained in the FASTQ file 106, plus the newly written forensic metadata. It is contemplated that, in other embodiments, the FASTF raw-format file 102 need not include all of the data from the FASTQ file 106.

As mentioned above, the FASTF raw-format file 102 includes forensic metadata (e.g., derived from the case file 110) in a first text line of each record of the file 102. In some embodiments, the forensic metadata may be in the form of one or more attribute-value pairs 300. In the illustrative embodiment of FIG. 3, the first text line of each record contains several attribute-value pairs 300, with each pair tab-separated. In some embodiments, the attribute-value pairs 300 may specify one or more of a file format, a unique case identifier, a unique sample identifier, a unique laboratory identifier, and a unique technician identifier associated with the file 102. Several illustrative attribute-value pairs 300 are set forth in Table 2 below, including possible permissible values and intended purposes.

TABLE 2

| Attribute | Permissible Values | Purpose |
|---|---|---|
| fileX.Y | X:= [0-9] | X refers to major releases and Y |
| | Y:=[0-9] | to minor releases |
| format | raw summary | Indicates whether each record |
| | | refers to raw nucleotide sequence |
| | | data (i.e., individual sequencer |
| | | reads) or summary STR data (i.e., |
| | | summary of multiple reads) |
| caseID | [A-Za-z0-9_.:-] | Unique case identity numbers for |
| | | case management |
| sampleID | [A-Za-z0-9_.:-] | Unique sample identity numbers for |
| | | case management |
| labID | [A-Za-z0-9_.:-] | Unique laboratory identity numbers |
| | | for case management |
| techID | [A-Za-z0-9_.:-] | Unique technician identity numbers |
| | | for case management |

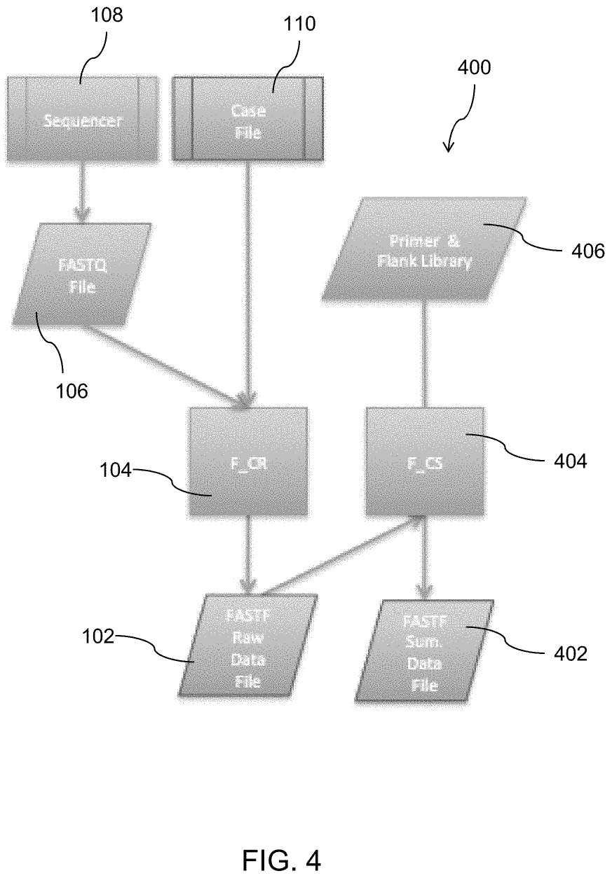

Referring now to FIG. 4, a method 400 of creating FASTF files containing summary nucleotide sequence data and related metadata is shown is as a simplified flow diagram (along with elements from the method 100, described above). The method 400 is intended for use nearer the end of the forensic DNA process and generates a FASTF summary-format file 402. While FASTF raw-format files 102 (like FASTQ files 106) may contain thousands to millions of records and be several gigaBytes in size, the FASTF summary-format file 402 is immensely more compact (e.g. on the order of kiloBytes), yet includes the information necessary to substantiate the results of an MPS-based allelotyping workflow. As described further below, the FASTF summary-format file 402 may contain a single record (i.e., four text lines) per locus for homozygous loci that do not exhibit stutter or artifacts. As another example, a FASTF summary-format file 402 for an 18 locus profile with 12 homozygous loci, 6 heterozygous loci, and half of the alleles showing a stutter response may contain just 45 records (i.e., 180 rows of human readable data).

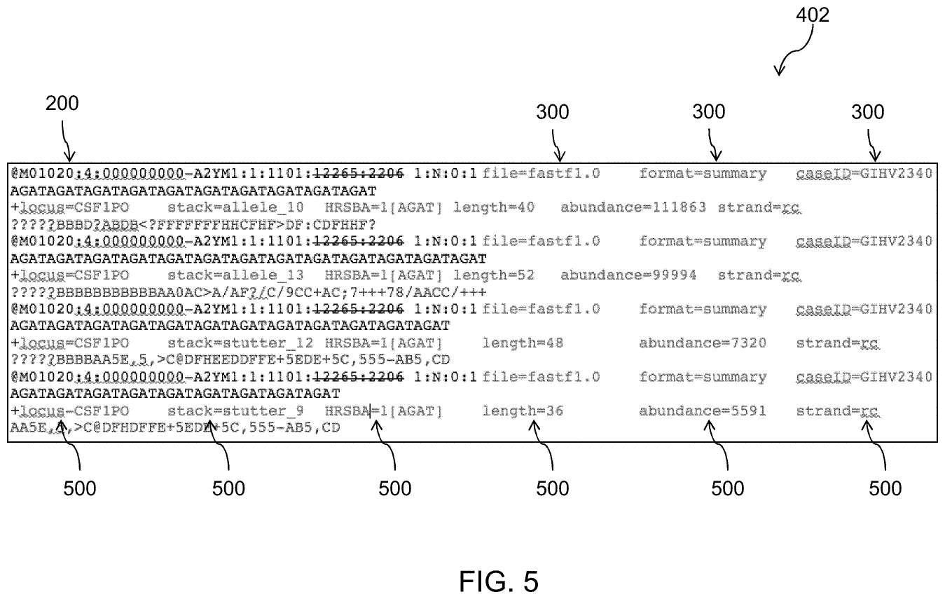

The F_CS algorithm 404 ingests a FASTF raw-format file 102 (e.g., generated by the F_CR algorithm 104) and the FASTF summary-format file 402. In order to perform this manipulation, the F_CS algorithm 404 may call a library 406 containing nucleotide sequences that may be used for locus identification and grooming operations. The nucleotide sequences stored in the library 406 may include 5′ primer sequences used to PCR amplify each STR locus, flanking sequences that are immediately 5′ and 3′ to each STR locus, and lists of canonical motifs found within each STR locus. The library 406 may be an updatable library 406, such that new information regarding the foregoing primer sequences, flanking sequences, and canonical motifs may be easily added and used by the F_CS algorithm 404. One illustrative example of a FASTF summary-format file 402 that may be output by the F_CS algorithm 404 is shown in FIG. 5 (showing the first sixteen lines, i.e. four records, of the file 402). After the method 400, the FASTF summary-format file 402 may be parsed into separate locus-specific files, may be graphed, may be used to generate CODIS-compliant allelotype records, and may be otherwise manipulated (e.g., using various FASTFtools utility algorithms), if desired.

Generally, the F_CS algorithm 404 generates the FASTF summary-format file 402 from the FASTF raw-format file 102 (or from a FASTQ file 106 or other raw file 102) by identifying all records of the raw file 102 that contain identical nucleotide sequence data representing a unique STR and grouping these records together to create the records of the summary file 402. After receiving the FASTF raw-format file 102 (or other raw file 102), the F_CS algorithm 404 examines each record in the raw file 102 to determine whether a portion of the nucleotide sequence data (e.g. in the second text line of each record) represents an STR associated with a locus. In some embodiments, this determination may involve identifying whether a portion of the nucleotide sequence data of each record represents a 5′ primer sequence used to amplify the locus. As noted above, the F_CS algorithm 404 may reference the library 406, which includes the primer sequences for each locus, when making this determination.

For each record in which a portion of the nucleotide sequence data is determined to represent an STR associated with a locus, the F_CS algorithm 404 will then place that portion of the nucleotide sequence data representing the STR associated into a locus-specific list. This may involve trimming away portions of the nucleotide sequence data that do not represent the STR. In particular, in some embodiments, the F_CS algorithm 404 may remove portions of the nucleotide sequence data that represent a flanking sequence. Once again, the F_CS algorithm 404 may reference the library 406, which contains lists of flanking sequences, when trimming the nucleotide sequence data.

After the portions of nucleotide sequence data representing STRs are each placed into locus-specific lists, the F_CS algorithm 404 will determine a number of occurrences within each locus-specific list of identical nucleotide sequence data representing a unique STR. In some embodiments, the F_CS algorithm 404 may compare the characters of each portion of nucleotide sequence data to all other portions in the same locus-specific list to find identical matches. This exact-matching scheme will result in a read count (i.e., a number of occurrences) for each even slightly different STR. For instance, where nucleotide sequence data reflects a single nucleotide polymorphism (SNP), the F_CS algorithm 404 will count this nucleotide sequence data as its own unique STR (and will not group it with a similar STR not exhibiting the SNP). It will be appreciated that this approach is quite different from prior alignment-based systems, which attempts to identify which reference sequence each portion of nucleotide sequence data most resembles. Such prior alignment-based system would typically lump the nucleotide sequence data reflecting the SNP in with similar sequences.

After the number of occurrences of each unique STR within each locus-specific list is determined, the F_CS algorithm 404 will select which nucleotide sequence data to write to the summary file 402. This determination is made based on some abundance threshold, which may take a number of forms and may be user-configurable, in some embodiments. For instance, the F_CS algorithm 404 may sort the unique STRs may relative number of occurrences and select a certain number of those at the top of the list for inclusion in the summary file 402. The remaining data, which essentially represents noise, will be discarded from the summary file 402 (but might still be retrieved from the raw file 102, if needed). In other embodiments, the abundance threshold may be embodied as a certain percentage threshold. For instance, all unique STRs that make up over 10% of the total reads for their locus-specific list might be included in the summary file 402. For each unique STR that is to be included in the summary file 402, the F_CS algorithm 404 will create one record in the summary file 402.

The FASTF summary-format file 402 retains the human readability feature of all FASTF files. This human readability allows direct use by adjudicating parties, who need evidentiary data presented in a way that is easily read and understood by lay audiences without the need for special software and bioinformatics expertise. As illustrated in FIG. 5, forensic metadata, in the form of attribute-value pairs 300, is presented in the first text line of each record of the file 402, appended to the unique sequence identifier 200 created by the MPS instrument 108. This information may be retained from the FASTF raw-format file 102 that is input to the F_CS algorithm 404. For illustration purposes, the x,y coordinates of the Illumina sequencing cluster are crossed out to indicate that these values would be replaced by null values because they are not relevant in the summary format. The second text line of each record of the FASTF summary-format file 402 comprises a number of characters representing nucleotide sequence data corresponding to one of the unique STRs selected by the F_CS algorithm 404 (i.e., an STR for which a read count in the sample exceeded an abundance threshold). The third text line of each record of the file 402 may comprise STR metadata summarizing the read records associated with the corresponding unique STR. In the illustrative embodiment of FIG. 5, the third text line of each record contains several attribute-value pairs 500, with each pair tab-separated. Several illustrative attribute-value pairs 500 are set forth in Table 3 below, including possible permissible values and intended purposes. The fourth text line of each record of the file 402 may comprise a number of characters representing average quality scores associated with the nucleotide sequence data of the second text line. These average quality scores may be a function of the quality scores of all reads summarized by that record and may be formatted as Phred quality scores. In other embodiments, the characters of the fourth text line may represent median quality scores associated with the nucleotide sequence data of the second text line.

TABLE 3

| Attribute | Permissible Values | Purpose |
|---|---|---|
| locus | X:= [0-9] | X refers to the number |
| | Y:=[0-9] | of full motif repeats, |
| | | Y refers to the number |
| | | of nucleotides in a |
| | | partial motif repeat |
| stack | allele_x[.y], | Identifies whether the |
| | stutter_x[.y], | read stack has been |
| | artifact_x[.y] | designated as an |
| | unassigned | allele, stutter, or an |
| | | artifact (x[.y] denotes |
| | | the ISFG allele |
| | | designation) |
| hrsba | [[ ]0-9 | Human readable sequence |
| | ACTGNURYSWKMBDHVN.-] | based allele designation |
| | | (IUPAC nucleotide |
| | | designations are used) |
| length | [0-9] | Length of nucleotide |
| | | sequence data |
| abundance | [0-9] | Read counts |
| | | corresponding to each |
| | | read stack |
| strand | isfgrc | Refers to the strand |
| | | represented by the |
| | | nucleotide sequence data |
| at | [0-9] | Refers to the analytic |
| | | threshold |

The portion of the FASTF summary-format file 402 illustrated in FIG. 5 exemplifies data from a single locus (namely, CSF1PO) for a case where the subject sample is single-source and is heterozygous. As reflected in FIG. 5, the subject is heterozygous 10,13 at this locus (denoted by the “stack=allele 10” and “stack=allele 13” value-attribute pairs 500) and the data exhibit stutter corresponding to allele lengths 9 and 12 (denoted by the “stack=stutter_9” and “stack=stutter 10” attribute-value pairs 500). In this case, only the DNA strand designated as the reverse complement of the ISO-standard strand is included (denoted by the “strand=rc” attribute-value pair 500). The read counts corresponding to each read stack is indicated by the “abundance” attribute-value pair 500. It will be appreciated that several ratios used in forensic analysis may be calculated from these values, including the stack height ratio (aka allele-coverage ratio) and the stutter ratio. A human-readable sequence-based allele (HRSBA) designation is also present. The HRSBA designation is reflected in the “hrsba” and “length” attribute-value pairs 500.

The HRSBA designation contained in the third text line of each record of the FASFT summary-format file 402 assists with the difficult task of reading repetitive DNA sequences and discerning the sometimes-subtle differences between them. HRSBA also provides a deterministic and non-arbitrary naming convention for sequence-defined alleles. Under current practice, STR alleles with identical nucleotide lengths but variant sequences are discriminated by denoting the allele length in International Society for Forensic Genetics (ISFG) X.Y format, followed by arbitrary notations such as letters, or prime or asterisk characters. These arbitrary notations fall short in two ways. First, they require the user to refer back to a lookup table for the actual sequence variations indicated by an asterisk or prime symbol. Second, without a complex and globally coordinated configuration management system, it is impossible to avoid inadvertent re-use of the same shorthand designations for different sequence variants by different laboratories. By contrast, the HRSBA designation disclosed herein has the advantage of determinism and clarity. By following a rule-based approach to generating HRSBA designations, every sequence variant will map to a unique HRSBA designation, allowing the underlying sequence to be easily derived from the HRSBA designation. The HRSBA designations may also be parsed by computer algorithms (e.g., FASTFtools utility algorithms) and rendered into alternative formats, including the original nucleotide sequence data.

In the illustrative embodiment, the HRSBA designation is reflected in two attribute-value pairs 500. First, the “hrsba” attribute-value pair 500 summarizes the STR sequence in the 5′ to 3′ direction, starting with the first nucleotide position in the STR sequence as nucleotide position 1. Nucleotide position numbers may be followed by the repeat motif bracketed (e.g., in square brackets) or by a single nucleotide code. It is contemplated that the HRSBA may use any type of punctuation to “bracket” a repeat motif. The nucleotide position refers to the position of the first nucleotide to the 3′ of the integer. In the case of a repeat motif, it refers to the first nucleotide of the motif. For example, a non-variant CSF1PO locus normally begins with the designation 1[AGAT], where the 5′ nucleotide A occupies nucleotide position number 1. The remaining three nucleotides (i.e., GAT) of the motif occupy sequence positions 2, 3 and 4, respectively. In the case of nucleotides not contained in a repeat motif, the positions of each nucleotide are denoted. For example, the TH01 allele 8.3 sequence listed by the National Institute of Standards and Technology (NIST) is as follows:

(SEQ ID NO: 1)
AATGAATGAATGAATGAATGATGAATGAATGAATG

In the illustrative embodiment, the HRSBA designation reflecting this sequence would be:

(SEQ ID NO: 1)
1[AATG]21ATG24[AATG] length = 35

This HRSBA designation indicates that the 5′ nucleotide A in the AATG repeat is the first nucleotide in the sequence, while the nucleotide A in the non-repeat motif ATG is in sequence position 21. Furthermore, the 5′ nucleotide A in the second repeat motif is in sequence position 24, and the total length of the sequence is 35, as indicated by the “length” attribute-value pair 500. Thus, the 3′ nucleotide G in the sequence is in sequence position 35. It will be appreciated from the foregoing that the HRSBA designation is unambiguous and deterministic for any sequence variant that might be encountered in actual sequencing. Any mutation can be accommodated, including complex ones such as segment inversions. Unlike the alignment-based methods typically used in current allelotyping processes, the HRSBA designation in not dependent upon reference sequences. Rather, it is reference independent and will not be affected by possible future changes to reference sequences.

As noted above, the value of the “length” attribute-value pair 500 denotes the total length of nucleotides comprising the STR sequence. This attribute-value pair 500 serves two purposes. First, it denotes the end of the STR sequence and therefore defines the number of repeats of the ending canonical motif of the sequence. Second, the length attribute-value pair 500 quickly identifies the allele length in nucleotides by visual inspection. Combined with the “locus” attribute-value pair, this information may be used to derive the ISFG allele designation, although this information is also encoded by the value of the “stack” attribute-value pair 500 in cases where the stack corresponds to an allele.

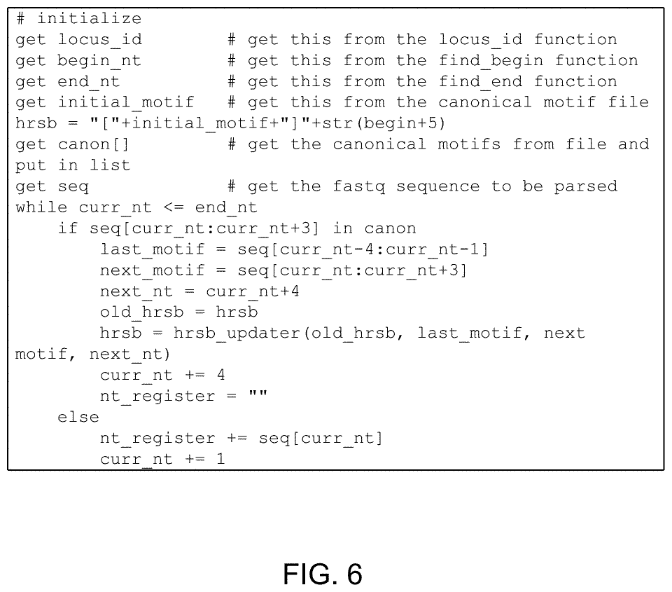

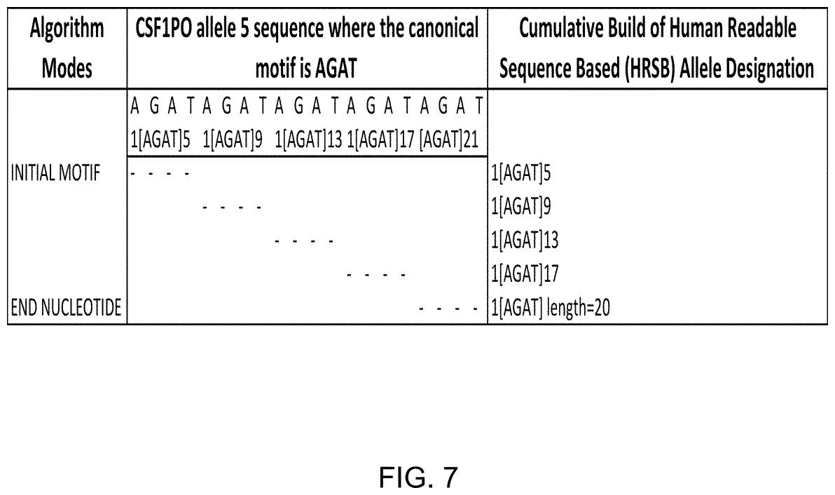

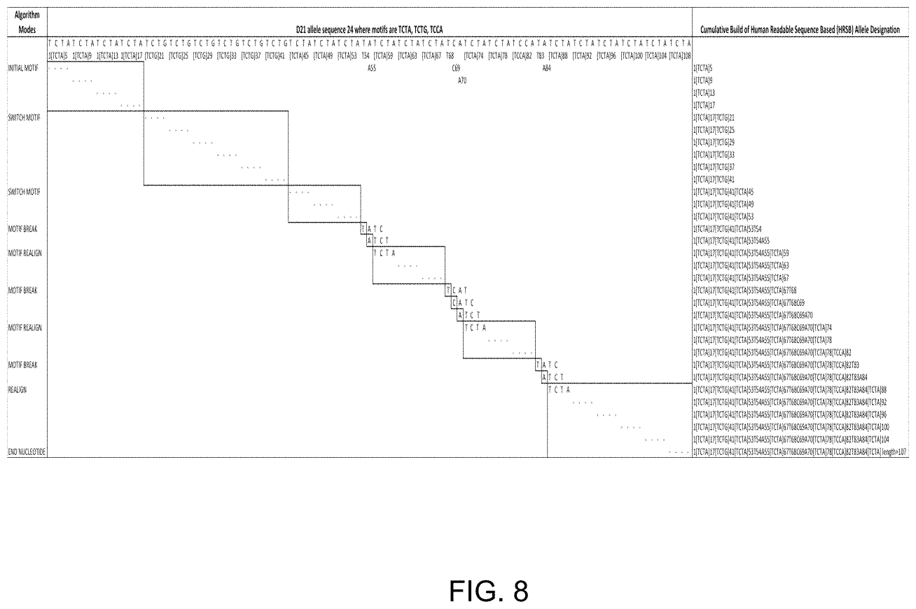

In the illustrative embodiment, the HRSBA designation described above is generated using a motif aware allellotyper (MAA) algorithm, which may form part of the F_CS algorithm 404. Pseudo-code for the MAA algorithm is presented in FIG. 6, while two examples of the operation of the MAA algorithm (described in detail below) are illustrated graphically in FIGS. 7 and 8. The MAA algorithm uses the portions of nucleotide sequence data determined to correspond to unique STRs, as described above, to generate the HRSBA designations corresponding to each unique STR. These HRSBA designations may then be included in each record of the FASTF summary-format file 402. The MAA algorithm leverages prior knowledge about the genetics and genomics of STR loci, particularly the facts that (1) the STR loci selected for use in forensic analysis exhibit a limited repertoire of repeat motifs within their alleles (i.e., the “canonical motifs” for that locus), (2) all alleles of a given forensic STR loci start with a specific initial repeat motif (i.e., a “canonical opening motif” for that locus), and (3) all alleles of a given forensic STR loci end with a specific final repeat motif (i.e., a “canonical ending motif” for that locus).

The illustrative embodiment of the MAA algorithm uses the ISFG convention that the initial repeat sequence motif be set equal to the first 5′ nucleotides that can define a repeat motif. Using this information, the deterministic HRSBA designation can be generated for each allele regardless of sequence. The MAA algorithm initializes on the occurrence of a canonical opening motif (see Table 4 below) immediately following the unique 5′ flanking sequence. In this embodiment, mutations in either of the 5′ flanking region or the initial repeat motif will result in allele dropout but not in an aberrant allele call, due to the deterministic property of the algorithm. In some embodiments, users may have the option of manually examining reads corresponding to loci exhibiting allele dropout.

TABLE 4

| | Canonical | Canonical | Set of |
|---|---|---|---|
| | Opening | Ending | Canonical |
| Locus | Motif | Motif | Motifs |
| CSF1PO | AGAT | AGAT | AGAT |
| FGA | TTTC | TTCC | TTTC, TTTTTT, CTTT, |
| | | | CTCC, TTCC, CTTC, |
| | | | CCTT |
| TH01 | AATG | AATG | AATG |
| TPOX | AATG | AATG | AATG |
| vWA | TCTA | TCTA | TCTA, TCTG, TCCA |
| D3S1358 | TCTA | TCTA | TCTA, TCTG |
| D5S818 | AGAT | AGAT | AGAT |
| D7S820 | GATA | GATA | GATA |
| D8S1179 | TCTA | TCTA | TCTA, TCTG |
| D13S317 | TATC | TATC | TATC |
| D16S539 | GATA | GATA | GATA |
| D18S51 | AGAA | AGAA | AGAA |
| D21S11 | TCTA | TCTA | TCTA, TCTG, TCCA |

Once initialized, the MAA algorithm utilizes a motif-aware 5′ to 3′ sliding window to parse the nucleotide sequence data of each allele. The sliding window follows two movement rules. First, if a canonical motif is present in the sliding window, a bracketing function is implemented for the canonical motif and then the sliding window moves four nucleotides to the right (3′). For the first instance of a canonical motif, the bracketing function adds the nucleotide bases in the sliding window to the HRSBA. In subsequent instances of the same canonical motif that is immediately adjacent to a prior instance, the bracketing function merely increments the nucleotide position to the immediate 3′ side of the canonical motif. Second, if a canonical motif is not found in the sliding window, the first nucleotide base in the window is pushed into a register and then the sliding window moves one position. This one-position movement of the four-nucleotide sliding window continues until the four-nucleotide sliding window encounters a canonical motif for that locus. When that occurs, the MAA algorithm again proceeds in four-position increments.

This movement of the sliding window continues until the final nucleotide of the STR allele sequence is encountered. The MAA algorithm performs a quality check to be sure the sequence ends in a canonical ending motif, generating a user alert otherwise. While the foregoing description uses the illustrative example of a four-nucleotide sliding window (to target tetramers), it is contemplated that the sliding window may be of any size. By way of example, the MAA algorithm may utilize a five-nucleotide sliding window to target pentameric repeats, such as those found in the pentaD and pentaE loci. In some embodiments, the MAA algorithm may switch to using a five-nucleotide sliding window when one of these loci is detected based on the uniquely-identifying 5′ primer sequence.

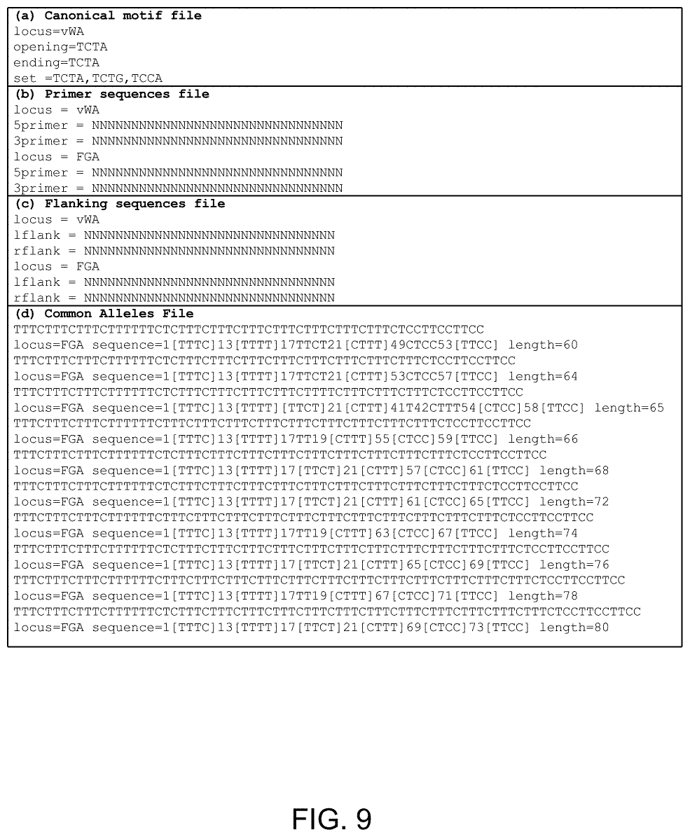

During operation, the MAA algorithm may call the library 406 described above to reference the canonical motifs for a particular locus. FIG. 9 shows a portion of one illustrative look-up table that may be contained in the library 406. As shown in FIG. 9 (part a), the library 406 may include a list of the canonical motifs associated with particular loci. The library 406 may also contain a list of primer sequences and flanking sequences for particular loci, as shown in FIG. 9 (parts b and c, respectively). In some embodiments, the library 406 may further contain lists of the HRSBA designations for common alleles (FIG. 9, part d). This feature may improve the speed of software implementing the MAA algorithm. By way of example, if a given allele sequence is identical to one of the “common alleles,” then a pre-computed HRSBA designation can simply be retrieved from a lookup table as opposed to constructing it de-novo with the MAA algorithm.

Reference to the external library 406 allows routine updating of information about forensic STR loci, without changes to the underlying software code implementing the MAA algorithm. The discovery of previously unknown allele sequences may be anticipated as the number of individuals sequenced increases. Some of these sequences may be observed frequently enough to be included in the library 406 as common alleles. Similarly, the canonical motifs used by the MAA algorithm may be updated in the future if additional repeat motifs are discovered for a given locus. Thus, the updatable library 406 allows software implementing the MAA algorithm to remain relevant and accurate.

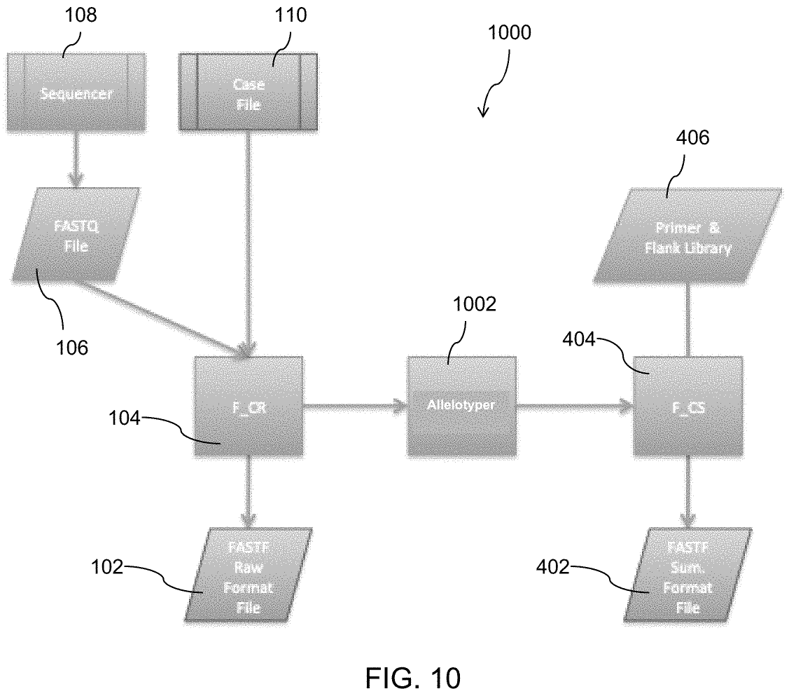

Referring now to FIG. 10, the methods 100, 400 described above may be used as pre- and post-processing in an allelotyping method 1000. In particular, the F_CR algorithm 104 and the F_CS algorithm 404 may function as pre-processing and post-processing, respectively, for an allelotyping process 1002 (e.g., as might be performed by other allelotyping software). Alternatively, the methods 100, 400 of creating FASTF files 102, 402 may be considered part of an allelotyping system themselves, particularly where FASTFtools utility algorithms and/or other algorithms are used as quality pre-processors and/or reporting post-processors.

While certain illustrative embodiments have been described in detail in the figures and the foregoing description, such an illustration and description is to be considered as exemplary and not restrictive in character, it being understood that only illustrative embodiments have been shown and described and that all changes and modifications that come within the spirit of the disclosure are desired to be protected. There are a plurality of advantages of the present disclosure arising from the various features of the apparatus, systems, and methods described herein. It will be noted that alternative embodiments of the apparatus, systems, and methods of the present disclosure may not include all of the features described yet still benefit from at least some of the advantages of such features. Those of ordinary skill in the art may readily devise their own implementations of the apparatus, systems, and methods that incorporate one or more of the features of the present disclosure.

1. An allelotyping method comprising:
using a massively parallel sequencing (MPS) instrument to read nucleotide sequences in a sample and to generate nucleotide sequence data quantifying each read of a nucleotide sequence in the sample;
determining, for each read by the MPS instrument, whether a portion of the generated nucleotide sequence data represents a short tandem repeat (STR) associated with a corresponding locus;
adding each portion of the nucleotide sequence data determined to represent an STR to a locus-specific list for the corresponding locus;
determining, for each locus-specific list, a number of occurrences within that locus-specific list of identical nucleotide sequence data representing a unique STR; and
identifying each unique STR for which the number of occurrences of identical nucleotide sequence data within the locus-specific list exceeds an abundance threshold as an allele of the corresponding locus for the sample.

2. The method of claim 1, further comprising amplifying nucleotide sequences in the sample using a PCR amplification process prior to using the MPS instrument to read nucleotide sequence in the sample.

3. The method of claim 1, wherein determining whether a portion of the generated nucleotide sequence data represents an STR associated with a corresponding locus comprises determining whether a portion of the generated nucleotide sequence data represents a primer sequence used to amplify the corresponding locus.

4. The method of claim 3, wherein determining whether a portion of the generated nucleotide sequence data represents a primer sequence used to amplify the corresponding locus comprises referencing an updateable library of primer sequences.

5. The method of claim 1, wherein adding each portion of the nucleotide sequence data determined to represent an STR to a locus-specific list for the corresponding locus comprises removing a portion of the nucleotide sequence data representing a flanking sequence.

6. The method of claim 5, wherein removing a portion of the nucleotide sequence data representing a flanking sequence comprises referencing an updateable library of flanking sequences.

7. The method of claim 1, wherein the abundance threshold is user-defined.

8. The method of claim 1, further comprising generating a text-based computer file including a record for each unique STR identified as an allele for the sample.

9. The method of claim 8, wherein generating the text-based computer file comprises, for each record, writing the nucleotide sequence data representing the corresponding unique STR to a second text line of the record.

10. The method of claim 9, wherein generating the text-based computer file further comprises, for each record, writing average quality scores for the nucleotide sequence data representing the corresponding unique STR to a fourth text line of the record.

11. The method of claim 10, wherein the average quality scores are formatted as average Phred quality scores.

12. The method of claim 9, wherein generating the text-based computer file further comprises, for each record, writing forensic metadata associated with the nucleotide sequence data representing the corresponding unique STR to a first text line of the record, wherein the forensic metadata identifies at least one of the sample and the MPS instrument.

13. The method of claim 9, wherein generating the text-based computer file further comprises, for each record, writing an attribute-value pair specifying the number of occurrences of identical nucleotide sequence data representing the corresponding unique STR to a third text line of the record.

14. The method of claim 9, wherein generating the text-based computer file further comprises, for each record, writing a human-readable sequence-based allele (HRSBA) designation that is deterministic of the corresponding unique STR to a third text line of the record.

15. The method of claim 14, further comprising generating the HRSBA designation from the nucleotide sequence data representing the corresponding unique STR, wherein generating the HRSBA designation comprises:
reading a plurality of nucleotide bases in a sliding window that moves along the nucleotide sequence data;
determining whether the plurality of nucleotide bases corresponds to a canonical motif of a locus associated with the corresponding unique STR;
adding the plurality of nucleotide bases to the HRSBA in response to determining that the plurality of nucleotide bases corresponds to a canonical motif of the locus and represents a first instance of the canonical motif; and
moving the sliding window by a plurality of positions in response to determining that the plurality of nucleotide bases corresponds to a canonical motif of the locus.

16. The method of claim 15, wherein generating the HRSBA designation further comprises:
adding only a first nucleotide base of the plurality of nucleotide bases to the HRSBA in response to determining that the plurality of nucleotide bases does not correspond to a canonical motif of the locus; and
moving the sliding window by one position in response to determining that the plurality of nucleotide bases does not correspond to a canonical motif of the locus.

17. The method of claim 15, wherein the sliding window moves along the nucleotide sequence data in a 5′ to 3′ direction.

18. The method of claim 15, wherein determining whether the plurality of nucleotide bases corresponds to a canonical motif of a locus associated with the corresponding unique STR comprises referencing an updatable library of canonical motifs of one or more loci.

19. The method of claim 15, wherein generating the HRSBA designation further comprises:
determining whether a final plurality of nucleotide bases of the nucleotide sequence corresponds to a canonical ending motif of a locus associated with the corresponding unique STR; and
generating a user alert in response to determining that the final plurality of nucleotide bases does not correspond to a canonical ending motif of the locus.

20. The method of claim 14, further comprising generating the HRSBA designation from the nucleotide sequence data representing the corresponding unique STR, wherein generating the HRSBA designation comprises referencing an updatable library associating common nucleotide sequences with corresponding HRSBA designations.
